(19) United States
(12) Patent Application Publication
(10) Pub. No.: US 2016/0296436 A1
(43) Pub. Date: Oct. 13, 2016

(54) COMPOSITIONS COMPRISING A SEBUM PUMP FILLER AND A HIGH QUANTITY OF MONO-ALCOHOL

(71) Applicant: L'OREAL, Paris (FR)

(72) Inventors: Pascal Arnaud, L'Hay Les Roses (FR); Anne-Marie Lezoray, Villeconin (FR); Damarys Braida, Paris (FR)

(21) Appl. No.: 15/101,726

(22) PCT Filed: Dec. 4, 2014
(86) PCT No.: PCT/EP2014/076656

§ 371 Date: Jun. 3, 2016

(30) Foreign Application Priority Data
Dec. 5, 2013 (FR) 13 62210

(51) Int. Cl. A61K 8/26; A61K 8/58; A61Q 1/02; A61Q 19/00; A61K 8/34

(52) U.S. Cl. CPC A61K 8/26; A61Q 19/008; A61K 8/34; A61Q 1/02; A61K 8/585; A61K 2800/87; A61K 2800/592

(57) ABSTRACT

The present invention relates to a cosmetic composition, comprising a cosmetically acceptable medium, containing a fatty phase and an aqueous phase, said aqueous phase comprising between 8% and 40% by weight of one or a plurality of mono-alcohol(s) comprising 2 to 8 carbon atoms, with respect to the total weight of said composition,
said composition comprising at least one filler absorbing and/or adsorbing sebum wherein the oil absorption capacity is greater than or equal to 1.5 ml/g, and
said composition comprising water in a quantity less than or equal to 5% by weight with respect to the total weight of said composition.

The present invention relates to skincare and/or makeup compositions. More specifically, the present invention relates to compositions comprising a sebum pump filler and a high quantity of mono-alcohol.

Consumers are increasingly looking for cosmetic makeup or skincare products that can be spread easily and quickly on the skin in the form of a deposit that should not be thick, but that instead merges as much as possible with the substrate.

It is known to a person skilled in the art that liquid products with a high oil content enable good spreading and skin penetration properties to be obtained.

Mention may be made, as examples of makeup products, of liquid foundations, liquid lip gloss, or in the field of skincare products, hydrating oily products and emollients or suntan oils.

This high oil content can, however, cause an oily and greasy sensation when applied, and lead to poor stability of the deposit on the skin over time.

It is therefore necessary to find a technical solution making it possible to obtain easy and agreeable spreading properties as well as properties of stability over time.

The aim of the invention is to meet these needs.

The aim of the present invention is thus that of providing a cosmetic composition with a satisfactory stability over time, while maintaining satisfactory application comfort.

The aim of the present invention is also that of providing a cosmetic composition giving a mattifying effect to the skin, with a good stability over time of said mattifying effect (for at least 1 hour, preferably at least 2 hours, advantageously at least 4 hours, for example at least 8 hours).

The aim of the present invention is thus also that of providing a cosmetic composition capable of being spread quickly and easily.

The aim of the present invention is also that of providing a cosmetic composition wherein the application properties do not involve a greasy sensation and wherein the deposit has a very good stability.

As such, the present invention relates to a cosmetic composition, comprising a cosmetically acceptable medium, containing a fatty phase and an aqueous phase, said aqueous phase comprising between 8% and 40% by weight of one or a plurality of mono-alcohol(s) comprising 2 to 8 carbon atoms, with respect to the total weight of said composition,

said composition comprising at least one filler absorbing and/or adsorbing sebum wherein the oil absorption capacity is greater than or equal to 1 ml/g, and
said composition comprising water in a quantity less than 30% by weight with respect to the total weight of said composition.

In particular, the present invention relates to a cosmetic composition, comprising a cosmetically acceptable medium, containing a fatty phase and an aqueous phase as defined above,

said composition comprising at least one filler absorbing and/or adsorbing sebum wherein the oil absorption capacity is greater than or equal to 1.5 ml/g, and
said composition comprising water in a quantity less than or equal to 5% by weight with respect to the total weight of said composition.

It was thus discovered, unexpectedly by the inventors, that the presence, in a liquid composition, of a high fatty phase content containing one or a plurality of sebum pump fillers, in association with a high mono-alcohol content, makes it possible to obtain products that spread easily and quickly, wherein the application properties do not involve a greasy sensation and wherein the deposit has a very good stability.

The inventors also discovered that said products are able to give a mattifying effect to the skin, with a good stability over time of said mattifying effect (for at least 1 hour, preferably at least 2 hours, advantageously at least 4 hours, for example at least 8 hours).

Fatty Phase

According to one embodiment, the fatty phase content of the compositions according to the invention ranges from 25% to 85%, preferably from 40% to 75% and even more preferably from 50% to 70% with respect to the total weight of the composition.

The fatty phase (or oil phase) of the compositions according to the invention comprises at least one oil. It may consist of a single oil or a mixture of a plurality of oils.

The term “oil” is intended to mean any fatty substance in liquid form at ambient temperature (20-25° C.) and at atmospheric pressure. These oils may be of animal, plant, mineral or synthetic origin.

More specifically, the fatty phase of the compositions according to the invention comprises at least one oil which may be volatile or non-volatile. Volatile oils are preferred.

In particular, according to the invention, the oil content is strictly greater than 1% by weight with respect to the total weight of the composition.

Volatile Oils

According to one embodiment, the fatty phase of the compositions according to the invention comprises at least one volatile oil. The fatty phase of the compositions according to the invention may comprise a mixture of a plurality of volatile oils.

The term “volatile oil” denotes an oil liable to evaporate on skin contact in less than one hour, at ambient temperature and atmospheric pressure. The volatile oil is a volatile cosmetic oil, liquid at ambient temperature, particularly having a vapor pressure different to zero, at ambient temperature and atmospheric pressure, particularly having a vapor pressure ranging from 0.13 Pa to 40000 Pa (10⁻³ at 300 mm Hg), and preferably ranging from 1.3 Pa to 13000 Pa (0.01 to 100 mm Hg), and preferentially ranging from 1.3 Pa to 1300 Pa (0.01 at 10 mm Hg).

Furthermore, the volatile oil generally has a boiling point, measured at atmospheric pressure, ranging from 150° C. to 260° C. and preferably ranging from 170° C. to 250° C.

To measure the evaporation rate mentioned above, 15 g of oil or an oil mixture to be tested is introduced into a crystallizer with a diameter of 7 cm, placed on a scale located in a large chamber of around 0.3 m³ with controlled temperature, at 25° C., and hygrometry, at 50% relative humidity. The liquid is left to evaporate freely, without stirring, by allowing ventilation with a fan (PAPST-MOTOREN, reference 8550 N, rotating at 2700 rpm) arranged vertically above the crystallizer containing said oil or said mixture, with the blades being directed toward the crystallizer and at a distance of 20 cm with respect to the crystallizer base. The mass of oil remaining in the crystallizer is measured at regular intervals. The evaporation rates are expressed in mg of oil evaporated per unit of surface (cm²) and per unit of time (minutes).

The volatile oils may be hydrocarbon, silicone or fluorinated oils.

According to the present invention, the term “hydrocarbon oil” denotes an oil containing mainly hydrogen and carbon atoms.

The term “silicone oil” denotes an oil comprising at least one silicon atom and particularly comprising at least one Si—O group.

The term “fluorinated oil” denotes an oil comprising at least one fluorine atom.

The volatile oils may be chosen from hydrocarbon oils having from 8 to 16 carbon atoms.

As a volatile hydrocarbon oil, mention may be made of C8-C16 branched alkanes, such as C8-C16 isoalkanes (also referred to as isoparaffins), isododecane, isohexadecane, or C9-C17 linear alkanes, such as dodecane (C12) and tetradecane (C14), marketed respectively under the references PARAFOL 12-97 and PARAFOL 14-97 by Sasol.

Of the volatile hydrocarbon oils, isododecane is preferred.

As a volatile silicone oil, mention may be made of linear or cyclic silicone oils having 2 to 7 silicon atoms, these silicones optionally comprising alkyl or alkoxy groups having 1 to 10 carbon atoms.

As examples, mention may be made of decamethyl cyclopentasiloxane, dodecamethyl cyclohexasiloxane and dodecamethyl pentasiloxane.

Of the volatile silicone oils, dodecamethyl pentasiloxane is preferred.

According to one embodiment, the fatty phase of the compositions according to the invention comprises a mixture of volatile oils, particularly volatile hydrocarbon and silicone oils. According to one particular embodiment, the fatty phase of the compositions according to the invention comprises a volatile hydrocarbon oil and a volatile silicone oil.

According to one preferred embodiment, the fatty phase of the compositions according to the invention comprises isododecane and dodecamethyl pentasiloxane.

Advantageously, the fatty phase comprises 40% to 100% by weight, preferably 60% to 98% by weight, and preferentially 80% to 95% by weight of volatile oil(s) with respect to the total weight of the fatty phase.

Non-Volatile Oils

According to one embodiment, the oil phase of the compositions according to the invention comprises at least one non-volatile oil. The oil phase of the compositions according to the invention may comprise a mixture of a plurality of non-volatile oils.

The term “non-volatile oil” denotes an oil remaining on the skin or keratin fiber at ambient temperature and atmospheric pressure. More specifically, a non-volatile oil has an evaporation rate strictly below 0.01 mg/cm²/min.

The non-volatile oils may, in particular, be chosen from non-volatile hydrocarbon, fluorinated and/or silicone oils.

Of the non-volatile hydrocarbon oils, mention may be made of natural oils or oils of natural origin such as paraffin oils or petroleum jelly, plant oils, such as sweet almond, jojoba and macadamia oils, mention may also be made of synthetic oils such as synthetic esters such as isodecyl neopentanoate or isononyl isononanoate or synthetic ethers such as dicaprylic ether.

Of the non-volatile silicone oils, mention may be made of polydimethylsiloxanes, phenylated silicones such as phenyltrimethicone or alkyldimethicones such as cetyl dimethicone.

Of the non-volatile fluorinated oils, mention may be made of fluorinated silicones and perfluoropolyethers.

According to one embodiment, the compositions according to the invention comprise phenyltrimethicone.

Advantageously, the fatty phase comprises 0% to 60% by weight, preferably 1% to 40% by weight, and preferentially 2% to 20% by weight of non-volatile oil(s) with respect to the total weight of the fatty phase.

Aqueous Phase

The composition according to the invention comprises an aqueous phase that may also be referred to as a hydro-alcoholic or alcoholic phase, comprising at least one mono-alcohol as defined above.

The alcoholic phase may also comprise water (hydro-alcoholic phase). A water suitable for the invention may be a floral water such as cornflower water and/or a mineral water such as Vittel water, Lucas water or La Roche Posay water and/or a spring water.

According to one embodiment, the quantity of water in the compositions according to the invention is less than 20% by weight and more preferentially less than 10% by weight and even more preferentially less than 5% by weight with respect to the total weight of the composition.

According to one particular embodiment, the composition according to the invention is anhydrous or contains less than 5% by weight of water, more preferably less than 2% by weight of water, even more preferentially less than 1% by weight of water, with respect to the total weight of said composition.

When the hydro-alcoholic phase of the compositions according to the invention does not comprise water, said phase shall be referred to as an “alcoholic phase”.

In addition to the lower mono-alcohol(s) defined above, this alcoholic or hydro-alcoholic phase may contain further alcohol(s), in particular polyethylene glycols having 6 to 80 ethylene oxide units; polyols such as propylene glycol, isoprene glycol, butylene glycol, glycerin, sorbitol, glycols such as propylene glycol, butylene glycol, dipropylene glycol, diethylene glycol, glycol ethers such as mono-, di- or tri-ethylene glyco alkyl(C₁-C₄), and mixtures thereof.

The alcoholic or hydro-alcoholic phase may also comprise stabilizers, such as sodium chloride, magnesium dichloride and magnesium sulfate.

The alcoholic or hydro-alcoholic phase may also comprise any water-soluble or water-dispersible compound compatible with an aqueous phase, such as gelling agents, thickeners, surfactants and mixtures thereof.

Mono-Alcohol

The compositions according to the invention comprise at least one mono-alcohol comprising 2 to 8 carbon atoms, in particular 2 to 6 carbon atoms and more particularly 2 to 4 carbon atoms.

The compositions according to the invention may comprise one or a plurality of mono-alcohol(s).

This mono-alcohol may be represented, for example, by the formula RₐOH, wherein Rₐ represents a linear or branched alkyl group, comprising 2 to 8 carbon atoms.

As examples of mono-alcohols, mention may be made of ethanol, isopropanol, propanol and butanol.

According to one embodiment, the compositions according to the invention comprise ethanol.

The quantity of mono-alcohol(s) is at least 8% by weight, and preferably at least 10% by weight with respect to the total weight of said composition.

According to one advantageous embodiment, the quantity of mono-alcohol(s) ranges from 10% to 40% by weight, preferably from 10% to 20% by weight and even more preferentially from 10% to 15% by weight with respect to the total weight of said composition.

The quantities by weight of mono-alcohol(s) correspond either to the quantity by weight of the mono-alcohol if the composition contains a single mono-alcohol or to the total quantity by weight of all of the mono-alcohols if the composition includes a mixture of a plurality of mono-alcohols.

According to one embodiment, the compositions according to the invention comprise 11% to 15%, preferably 11.4% to 15%, by weight of mono-alcohol(s), preferably ethanol, with respect to the total weight of said composition.

Filler Absorbing and/or Adsorbing Sebum

A composition according to the invention comprises at least one filler absorbing and/or adsorbing sebum capable of absorbing and/or adsorbing an oil or a liquid fat such as sebum (from skin), for example. This filler is also called a “sebum pump filler”.

In particular, the filler used according to the invention has an oil absorption capacity greater than or equal to 1.5 ml/g.

This filler absorbing and/or adsorbing sebum may further advantageously have a BET specific surface area greater than or equal to 300 m²/g, preferably greater than 500 m²/g, and preferentially greater than 600 m²/g, and particularly less than 1500 m²/g.

The “BET specific surface area” is determined using the BET (BRUNAUER-EMMET -TELLER) method described in “The Journal of the American Chemical Society”, vol. 60, page 309, February 1938 and corresponding to international standard ISO 5794/1 (Appendix D). The BET specific surface area is the total specific area of the particles in question.

The filler in question according to the invention is thus characterized in that it has an oil take-up greater than or equal to 1.5 ml/g, particularly ranging from 1.5 ml/g to 20 ml/g, or ranging from 1.5 ml/g to 15 ml/g. Preferably, it has an oil take-up greater than or equal to 2 ml/g, particularly ranging from 2 ml/g to 20 ml/g, or ranging from 2 ml/g to 15 ml/g.

This oil take-up corresponding to the quantity of oil absorbed and/or adsorbed by the filler may be characterized by the Wet Point measurement using the method described hereinafter.

Filler Oil Take-Up Measurement Method:

The oil take-up of a powder is measured using the method for determining oil take-up of powder described in the standard NF T 30-022. It corresponds to the quantity of oil adsorbed on the available surface of the filler by measuring the Wet Point.

A quantity m (in grams) of powder between approximately 0.5 g and 5 g (the quantity is dependent on the powder density) is placed on a glass plate and isononyl isononanoate is then added drop by drop.

After adding 4 to 5 drops of isononyl isononanoate, isononyl isononanoate is incorporated in the filler using a spatula and the addition of isononyl isononanoate is continued until isononyl isononanoate powder conglomerates are formed. From this stage, isononyl isononanoate is added at a rate of one drop at a time and the mixture is then triturated with the spatula. The addition of isononyl isononanoate is stopped when a firm and smooth paste is obtained. This paste should be allowed to spread on the glass plate without splitting or lump formation. The volume Vs (expressed in ml) of isononyl isononanoate used is then noted.

The oil take-up corresponds to the ratio Vs/m.

The filler absorbing and/or adsorbing sebum according to the invention (or sebum pump filler) may be organic or inorganic.

In particular, a composition according to the invention comprises a sebum pump filler chosen from silicas, silica silylates (particularly hydrophobic silica aerogels), polyamide powders (particularly nylon-6), acrylic polymer powders, particularly methyl polymethacrylate, methyl polymethacrylate/ethylene glycol dimethacrylate, allyl polymethacrylate/ethylene glycol dimethacrylate, ethylene glycol dimethacrylate/lauryl methacrylate copolymer; perlites; magnesium carbonate and mixtures thereof.

According to one embodiment, the sebum pump filler according to the invention is chosen from silicas, silica silylates (particularly hydrophobic silica aerogels), acrylic polymer powders, particularly methyl polymethacrylate, methyl polymethacrylate/ethylene glycol dimethacrylate, allyl polymethacrylate/ethylene glycol dimethacrylate, ethylene glycol dimethacrylate/lauryl methacrylate copolymer; perlites; magnesium carbonate and mixtures thereof.

According to a further embodiment, the sebum pump filler according to the invention is chosen from silicas, polyamide powders (particularly nylon-6), acrylic polymer powders, particularly methyl polymethacrylate, methyl polymethacrylate/ethylene glycol dimethacrylate, allyl polymethacrylate/ethylene glycol dimethacrylate, ethylene glycol dimethacrylate/lauryl methacrylate copolymer; perlites; magnesium carbonate and mixtures thereof.

According to a further embodiment, the sebum pump filler according to the invention is chosen from silicas, acrylic polymer powders, particularly methyl polymethacrylate, methyl polymethacrylate/ethylene glycol dimethacrylate, allyl polymethacrylate/ethylene glycol dimethacrylate, ethylene glycol dimethacrylate/lauryl methacrylate copolymer; perlites; magnesium carbonate and mixtures thereof.

Those skilled in the art would select from the abovementioned materials, the filler(s) having an oil take-up greater than or equal to 1 ml/g, particularly greater than or equal to 1.5 ml/g, preferably greater than or equal to 2 ml/g and, as such, suitable for the invention.

Advantageously, the filler absorbing and/or adsorbing sebum may be a powder coated with a hydrophobic treatment agent.

The hydrophobic treatment agent may particularly be chosen from fatty acids such as stearic acid; metal soaps such as aluminum dimyristate, aluminum hydrogenated tallow glutamate; amino acids; N-acylated amino acids and salts thereof; lecithin; isopropyl triisostearyl titanate; mineral waxes and mixtures thereof.

The N-acylated amino acids may comprise an acyl group having 8 to 22 carbon atoms, such as for example a 2-ethyl hexanoyl, caproyl, lauroyl, myristoyl, palmitoyl, stearoyl or cocoyl group. The salts of these compounds may be aluminum, magnesium, calcium, zirconium, zinc, sodium, potassium salts. The amino acid may be for example lysine, glutamic acid or alanine.

The term alkyl mentioned in the above-mentioned compounds particularly denotes an alkyl group having 1 to 30 carbon atoms, preferably having 5 to 16 carbon atoms.

Examples of fillers according to the invention, i.e. having an oil take-up greater than or equal to 1 ml/g, in particular 1.5 ml/g, are described hereinafter, with the oil take-up value thereof measured according to the protocol described above.

As silica powder, mention may be made of:
porous silica microspheres, particularly those sold under the trade name “SUNSPHERE H53”, “SUNSPHERE H33” (oil take-up equal to 3.70 ml/g) by ASAHI GLASS; MSS-500-3H by KOBO; or
amorphous silica microspheres coated with polydimethylsiloxane, particularly those sold under the trade name “SA SUNSPHERE H 33” (oil take-up equal to 2.43 ml/g); or
silica silylate powders, particularly those sold under the trade name Dow Corning VM-2270 Aerogel Fine Particles by Dow Corning (oil take-up equal to 10.40 ml/g); or
hollow amorphous silica particles, particularly those sold under the trade name Silica Shells by Kobo (oil take-up equal to 5.50 ml/g); or
precipitated silica powders, surface-treated with a mineral wax, such as precipitated silica treated with a polyethylene wax and particularly those sold under the trade name ACEMATT OR 412 by EVONIK DEGUSSA (oil take-up equal to 3.98 ml/g).

According to one embodiment, it is possible to use, according to the invention, as silica powder: porous silica microspheres, particularly those sold under the trade name “SUNSPHERE H53”, “SUNSPHERE H33” (oil take-up equal to 3.70 ml/g) by ASAHI GLASS; MSS-500-3H by KOBO; or amorphous silica microspheres coated with polydimethylsiloxane, particularly those sold under the trade name “SA SUNSPHERE H 33” (oil take-up equal to 2.43 ml/g); or hollow amorphous silica particles, particularly those sold under the trade name Silica Shells by Kobo (oil take-up equal to 5.50 ml/g); or precipitated silica powders, surface-treated with a mineral wax, such as precipitated silica treated with a polyethylene wax and particularly those sold under the trade name ACEMATT OR 412 by EVONIK DEGUSSA (oil take-up equal to 3.98 ml/g).

As acrylic polymer powder, mention may be made of:
porous methyl polymethacrylate/ethylene glycol dimethacrylate spheres sold under the trade name Microsponge 5640, by Cardinal Health technologies (oil take-up equal to 1.55 ml/g); or
ethylene glycol dimethacrylate/lauryl methacrylate copolymer powders, particularly those sold under the trade name POLYTRAP 6603 by DOW CORNING (oil take-up equal to 6.56 ml/g).

As polyamide powder, mention may be made of nylon-6 powder, particularly that sold under the trade name POMP610 by UBE INDUSTRIES (oil take-up equal to 2.02 ml/g).

As perlite powder, mention may particularly be made of that sold under the trade name OPTIMAT 2550 OR by WORLD MINERALS (oil take-up equal to 2.4 ml/g).

As magnesium carbonate powder, mention may particularly be made of that sold under the trade name TIPO CARBOMAGEL by BUSCHLE & LEPPER (oil take-up equal to 2.14 ml/g).

The particularly preferred filler absorbing and/or adsorbing sebum is a perlite powder or a silica powder, and more particularly a silica powder having an oil take-up at least equal to 3.70 ml/g and particularly those sold under the trade name “SUNSPHERE H 33” by ASAHI GLASS, and under the trade name Dow Corning VM-2270 Aerogel Fine Particles by Dow Corning.

The filler(s) having an oil take-up greater than or equal to 1 ml/g, particularly greater than or equal to 1.5 ml/g, otherwise known as sebum pump fillers according to the invention, may be present in a composition according to the invention at a content ranging from 0.05% to 20% by weight, preferably from 1% to 10% by weight, and more preferably from 1% to 6% by weight, with respect to the total weight of the composition.

According to one embodiment, the filler absorbing and/or adsorbing sebum is in the fatty phase.

Physiologically Acceptable Medium

In addition to the compounds indicated above, a composition according to the invention comprises a physiologically acceptable medium.

The term “physiologically acceptable medium” is intended to denote a medium that is particularly suitable for the application of a composition of the invention to the skin or the lips.

The physiologically acceptable medium is generally suitable for the nature of the substrate to which the composition is to be applied, and also for the way in which the composition is to be packaged.

The compositions according to the invention may be anhydrous or in the form of a water-in-oil (W/O), oil-in-water (O/W) emulsion or even a multiple emulsion.

When the product is in the form of an emulsion, the W/O form is preferred.

Lipophilic Structuring Agent

A composition according to the invention may also comprise at least one agent for structuring a liquid fatty phase, selected from a wax, a pasty compound, and mixtures thereof.

In particular, a wax suitable for the invention may especially be selected from waxes of animal, plant, mineral and synthetic origin, and mixtures thereof.

By way of examples of waxes that can be used according to the invention, mention may be made of:
waxes of animal origin, such as beeswax, spermaceti, lanolin wax and lanolin derivatives, plant waxes such as carnauba wax, candelilla wax, ouricury wax, Japan wax, cocoa butter, cork fiber wax or sugarcane wax,
mineral waxes, for example paraffin wax, petroleum jelly wax, lignite wax or microcrystalline waxes or ozokerites,
synthetic waxes, among which are polyethylene waxes, and waxes obtained by Fisher-Tropsch synthesis,
silicone waxes, in particular substituted linear polysiloxanes; mention may, for example, be made of silicone polyether waxes, alkyl dimethicones or alkoxy dimethicones having 16 to 45 carbon atoms, alkyl methicones such as the C₃₀-C₄₅ alkyl methicone sold under the trade name “AMS C 30” by DOW CORNING,
hydrogenated oils solid at 25° C. such as hydrogenated castor oil, hydrogenated jojoba oil, hydrogenated palm oil, hydrogenated tallow or hydrogenated coconut oil, and fatty esters that are solid at 25° C., such as C₂₀-C₄₀ alkyl stearate sold under the trade name “KESTER WAX K82H” by KOSTER KEUNEN,
and/or mixtures thereof.

Preferably, polyethylene waxes, microcrystalline waxes, carnauba waxes, hydrogenated jojoba oil, candelilla waxes, beeswaxes and/or mixtures thereof will be used.

A composition according to the invention may comprise at least one pasty compound.

The presence of a pasty compound may make it possible to advantageously confer improved comfort when a composition of the invention is deposited on keratin fibers.

Such a compound may advantageously be selected from lanolin and derivatives thereof; polymeric or non-polymeric silicone compounds; polymeric or non-polymeric fluorine compounds; vinyl polymers, in particular olefin homopolymers; olefin copolymers; hydrogenated diene homopolymers and copolymers; linear or branched and homo- or copolymeric oligomers of alkyl (meth)acrylates preferably having a C₈-C₃₀ alkyl group; homo- and copolymeric oligomers of vinyl esters having C₈-C₃₀ alkyl groups; homo- and copolymeric oligomers of vinyl ethers having C₈-C₃₀ alkyl groups; liposoluble polyethers resulting from polyetherification between one or a plurality of C₂-C₁₀₀, in particular, C₂-C₅₀ diols; fatty acid or alcohol esters; and mixtures thereof.

Of the esters, mention may particularly be made of:
the esters of an oligomeric glycerol, especially the esters of diglycerol, for instance polyglyceryl-2 triisostearate, the condensates of adipic acid and of glycerol, for which a portion of the hydroxyl groups of the glycerols have reacted with a mixture of fatty acids, such as stearic acid, capric acid, stearic acid and isostearic acid and 12-hydroxystearic acid, such as, in particular, those marketed under the trade mark Softisan 649 by Sasol or such as bisdiglyceryl polyacyladipate-2; arachidyl propionate marketed under the trade mark Waxenol 801 by Alzo; phytosterol esters; triglycerides of fatty acids and derivatives thereof, such as hydrogenated cocoglycerides; non-cross-linked polyesters resulting from polycondensation between a linear or branched C₄-C₅₀ dicarboxylic acid or polycarboxylic acid and a C₂-C₅₀ diol or polyol; aliphatic esters of an ester resulting from the esterification of an aliphatic hydroxycarboxylic acid ester with an aliphatic carboxylic acid; polyesters resulting from the esterification, with a polycarboxylic acid, of an aliphatic hydroxycarboxylic acid ester, said ester comprising at least two hydroxyl groups, such as the products Risocast DA-H®, and Risocast DA-L®; and mixtures thereof.

The structuring agent(s) may be present in a composition according to the invention in a content ranging from 0.1% to 30% by weight of agents, preferably from 0.5% to 20% by weight, with respect to the total weight of the composition.

Thickeners

Depending on the fluidity of the composition sought, one or a plurality of thickeners or gelling agents may be incorporated into a composition according to the invention.

A thickener or gelling agent suitable for the invention may be hydrophilic, i.e. water-soluble or water-dispersible.

As hydrophilic gelling agents, mention may in particular be made of water-soluble or water-dispersible thickening polymers. Said polymers may in particular be selected from: modified or unmodified carboxyvinyl polymers, such as the products sold under the trade name Carbopol (CTFA name: carbomer) by Goodrich; polyacrylates and polymethacrylates, such as the products sold under the trade names Lubrajel and Norgel by GUARDIAN or under the trade name Hispagel by HISPANO CHIMICA; polyacrylamides; 2-acrylamido-2-methylpropanesulfonic acid polymers and copolymers, which are optionally cross-linked and/or neutralized, such as the poly(2-acrylamido-2-methylpropanesulfonic acid) sold by CLARIANT under the trade name “Hostacerin AMPS” (CTFA name: ammonium polyacryldimethyltauramide); cross-linked anionic acrylamide/AMPS copolymers, in the form of a W/O emulsion, such as those marketed under the name SEPIGEL 305 (CTFA name: Polyacrylamide/C13-14 lsoparaffin/Laureth-7) and under the name SIMULGEL 600 (CTFA name: Acrylamide/Sodium acryloyldimethyltaurate copolymer/lsohexadecane/Polysorbate 80) by SEPPIC; polysaccharide biopolymers, such as xanthan gum, guar gum, carob gum, gum acacia, scleroglucans, chitin derivatives and chitosan derivatives, carrageenans, gellans, alginates, or celluloses such as microcrystalline cellulose, carboxymethylcellulose, hydroxymethylcellullose and hydroxypropylcellulose; and mixtures thereof.

A thickener or gelling agent suitable for the invention may be lipophilic. It may be mineral or organic.

As lipophilic thickeners, mention may, for example, be made of modified clays, such as modified magnesium silicate (Bentone gel VS38 from RHEOX), modified hectorites such as hectorite modified with a C₁₀ to C₂₂ fatty acid ammonium chloride, for instance hectorite modified with distearyldimethylammonium chloride such as, for example, that marketed under the trade name Bentone 38V® by ELEMENTIS or that marketed under the trade name “Bentone 38 CE” by RHEOX or that marketed under the trade name Bentone Gel V5 5V by ELEMENTIS.

The polymeric organic lipophilic gelling agents are, for example, partially or totally cross-linked elastomeric organopolysiloxanes with a three-dimensional structure, such as those marketed under the trade names KSG6®, KSG16® and KSG18® by SHIN-ETSU, Trefil E-505C® Trefil E-506C® by DOW-CORNING, Gransil SR-CYC®, SR DMF10@, SR-DC556@, SR SCYC Gel®, SR DMF 10 Gel® and SR DC 556 Gel® by GRANT INDUSTRIES, SF 1204® and JK 113® by GENERAL ELECTRIC; polyimide-type polycondensates resulting from condensation between a dicarboxylic acid containing at least 32 carbon atoms and an alkylene diamine, and in particular ethylene diamine, wherein the polymer comprises at least one terminal carboxylic acid group esterified or amidified with at least one monoalcohol or one monoamine containing from 12 to 30 carbon atoms, and linear and saturated, and in particular ethylenediamine/stearyl dilinoleate copolymers such as that marketed under the trade name Uniclear 100 VG® by ARIZONA CHEMICAL; galactomannans comprising from one to six, and in particular from two to four, hydroxyl groups per monosaccharide, substituted with a saturated or unsaturated alkyl chain, such as guar gum alkylated with C₁ to C₆ alkyl chains, and in particular C₁ to C₃ alkyl chains, and mixtures thereof. Block copolymers of the “diblock”, “triblock” or “radial” type, of the polystyrene/polyisoprene or polystyrene/polybutadiene type, such as those marketed under the trade name Luvitol HSB® by BASF, of the polystyrene/copoly(ethylene-propylene) type, such as those marketed under the name Kraton® by SHELL CHEMICAL CO or of the polystyrene/copoly(ethylene-butylene) type, mixtures of triblock and radial (star) copolymers in isododecane, such as those marketed by PENRECO under the trade name Versagel® for instance the mixture of butylene/ethylene/styrene triblock copolymer and of ethylene/propylene/styrene star copolymer in isododecane (Versagel M 5960).

Of the lipophilic gelling agents that can be used in a cosmetic composition of the invention, mention may also be made of esters of dextrin and of a fatty acid, such as dextrin palmitates, in particular such as those marketed under the trade names Rheopearl TL® or Rheopearl KL® by CHIBA FLOUR, hydrogenated plant oils, such as hydrogenated castor oil, fatty alcohols, in particular C₈ to C₂₆, and more particularly C₁₂ to C₂₂ fatty alcohols, for instance myristyl alcohol, cetyl alcohol, stearyl alcohol and behenyl alcohol.

According to one embodiment, a composition according to the invention may comprise thickeners in an active substance content of 0.01% to 40% by weight, especially from 0.1% to 20% by weight, in particular from 0.3% to 15% by weight, with respect to the total weight of the composition.

According to one preferred embodiment, the composition comprises at least one lipophilic thickener, in particular at least one modified hectorite, such as a hectorite modified by a C₁₀ to C₂₂ fatty acid ammonium chloride, advantageously in a content ranging from 0.1% to 5% by weight, in particular 0.5% to 2% by weight of active substance with respect to the total weight of said composition.

The composition may also include at least one powder substance, in particular chosen from powder dyes and fillers. According to one particular embodiment, the composition according to the invention comprises at least pigments and at least one filler. The powder substance content will generally range from 5% to 30% by weight, and in particular from 10% to 20% by weight with respect to the total weight of said composition.

Dyes

A composition according to the invention may also comprise at least one dye, in particular a powder dye.

The dye(s) may be present with a content ranging from 2% to 25% by weight, in particular from 5% to 20% by weight, and preferably from 8% by 15% by weight with respect to the total weight of said composition.

A cosmetic composition according to the invention may advantageously incorporate at least one dye selected from organic or inorganic dyes, in particular such as pigments or nacres conventionally used in cosmetic compositions, liposoluble or water-soluble coloring agents, materials with a specific optical effect, and mixtures thereof.

The term “pigments” should be understood to mean white or colored, mineral or organic particles, which are insoluble in an aqueous solution and are intended for coloring and/or opacifying the resulting film.

The pigments may be present in a proportion of 0.1% to 40% by weight, such as from 1% to 30% by weight, or from 5% to 15% by weight, with respect to the total weight of the cosmetic composition.

As mineral pigments suitable for use in the invention, mention may be made of titanium, zirconium or cerium oxides, and also zinc, iron or chromium oxides, ferric blue, manganese violet, ultramarine blue and chromium hydrate. Preferably, the composition according to the invention comprises at least titanium oxides and iron oxides.

The pigment may also be a pigment having a structure that may be, for example, of sericite/brown iron oxide/titanium dioxide/silica type. Such a pigment is marketed, for example, under the reference COVERLEAF NS or JS by CHEMICALS AND CATALYSTS and has a contrast ratio of around 30.

The dye may also comprise a pigment having a structure that may, for example, be of the type of silica microspheres containing iron oxide. An example of a pigment having this structure is marketed by MIYOSHI under the name PC BALL PC-LL-100 P, and this pigment consists of silica microspheres containing yellow iron oxide.

The term “nacres” should be understood to mean iridescent or non-iridescent colored particles of any shape, which are in particular produced by certain mollusks in their shell or else are synthesized and which exhibit a color effect by optical interference.

The nacres may be selected from pearlescent pigments such as titanium mica coated with iron oxide, titanium mica coated with bismuth oxychloride, titanium mica coated with chromium oxide, titanium mica coated with an organic dye, and pearlescent pigments based on bismuth oxychloride. This may also involve mica particles at the surface whereof are superposed at least two successive layers of metal oxides and/or of organic dyes.

By way of example of nacres, mention may also be made of natural mica coated with titanium oxide, with iron oxide, with natural pigment or with bismuth oxychloride.

Among the nacres available on the market, mention may be made of the TIMICA, FLAMENCO and DUOCHROME nacres (based on mica) marketed by ENGELHARD, the TIMIRON nacres marketed by MERCK, the nacres based on mica, PRESTIGE, marketed by ECKART and the nacres based on synthetic mica, SUNSHINE, marketed by SUN CHEMICAL.

The nacres may more particularly have a yellow, pink, red, bronze, orange, brown, gold and/or copper color or glint.

By way of illustration of nacres which can be used in the context of the invention, mention may, in particular, be made of the gold nacres marketed, in particular, by ENGELHARD, under the name Brilliant gold 212G (Timica), Gold 222C (Cloisonne), Sparkle gold (Timica), Gold 4504 (Chromalite) and Monarch gold 233X (Cloisonne); the bronze nacres, marketed, in particular, by MERCK under the name Bronze fine (17384) (Colorona) and Bronze (17353) (Colorona) and by ENGELHARD under the name Super bronze (Cloisonne); the orange nacres, in particular, marketed by ENGELHARD under the name Orange 363C (Cloisonne) and Orange MCR 101 (Cosmica) and by MERCK under the name Passion orange (Colorona) and Matte orange (17449) (Microna); the brown-hued nacres marketed in particular by ENGELHARD under the name Nu-antique copper 340XB (Cloisonne) and Brown CL4509 (Chromalite); the copper-glint nacres marketed in particular by ENGELHARD under the name Copper 340A (Timica); the red-glint nacres marketed in particular by MERCK under the name Sienna fine (17386) (Colorona); the yellow-glint nacres marketed in particular by ENGELHARD under the name Yellow (4502) (Chromalite); the gold-glint red-hued nacres marketed in particular by ENGELHARD under the name Sunstone G012 (Gemtone); the pink nacres marketed in particular by ENGELHARD under the name Tan opal G005 (Gemtone); the gold-glint black nacres marketed in particular by ENGELHARD under the name Nu-antique bronze 240 AB (Timica), the blue nacres marketed in particular by MERCK under the name Matte blue (17433) (Microna), the silver-glint white nacres marketed in particular by MERCK under the name Xirona Silver and the green-gold and pinkish orangish nacres marketed in particular by MERCK under the name Indian summer (Xirona) and mixtures thereof.

The cosmetic composition according to the invention may also comprise water-soluble or liposoluble colorants. The liposoluble colorants are, for example, Sudan Red, DC Red 17, DC Green 6, β-carotene, soybean oil, Sudan Brown, DC Yellow 11, DC Violet 2, DC Orange 5, and Quinoline Yellow. The water-soluble colorants are, for example, beetroot juice and caramel.

The cosmetic composition according to the invention may also contain at least one material with a specific optical effect.

This effect is different from a simple conventional hue effect, i.e. a unified and stabilized effect of the kind produced by conventional dyes, such as, for example, monochromatic pigments. For the purpose of the invention, the term “stabilized” signifies absence of an effect of variability of color with the angle of observation or in response to a temperature change.

For example, this material may be selected from particles having a metallic glint, goniochromatic coloring agents, diffracting pigments, thermochromatic agents, optical brighteners, and also fibers, in particular of the interference type. Of course, these various materials may be combined so as to provide the simultaneous manifestation of two effects, or even a new effect in accordance with the invention.

The metallic-glint particles that can be used in the invention are in particular chosen from:
particles of at least one metal and/or of at least one metal derivative,
particles comprising a single-substance or multi-substance, organic or mineral substrate, at least partially coated with at least one metallic-glint layer comprising at least one metal and/or at least one metal derivative, and
mixtures of said particles.

Among the metals that may be present in said particles, mention may, for example, be made of Ag, Au, Cu, Al, Ni, Sn, Mg, Cr, Mo, Ti, Zr, Pt, Va, Rb, W, Zn, Ge, Te, Se and mixtures or alloys thereof. Ag, Au, Cu, Al, Zn, Ni, Mo, Cr and mixtures or alloys thereof (for example, bronzes and brasses) are preferred metals.

The term “metal derivatives” denotes compounds derived from metals, in particular oxides, fluorides, chlorides and sulfides.

By way of illustration of these particles, mention may be made of aluminum particles, such as those marketed under the trade names STARBRITE 1200 EAC® by SIBERLINE and METALURE® by ECKART.

Mention may also be made of metal powders of copper or of alloy mixtures, such as the references 2844 marketed by RADIUM BRONZE, metal pigments, such as aluminum or bronze, for instance those marketed under the trade name ROTOSAFE 700 by ECKART, silica-coated aluminum particles marketed under the trade name VISIONAIRE BRIGHT SILVER bye ECKART and metal alloy particles, such as silica-coated bronze (copper and zinc alloy) marketed under the trade name Visionaire Bright Natural Gold by Eckart.

The particles in question may also be particles comprising a glass substrate, such as those marketed by NIPPON SHEET GLASS under the trade name MICROGLASS METASHINE.

The goniochromatic coloring agent may be selected, for example, from multilayer interference structures and liquid-crystal coloring agents.

Examples of symmetrical multilayer interference structures that may be used in compositions prepared according to the invention are, for example, the following structures: Al/SiO₂/Al/SiO₂/Al, pigments having this structure being marketed by DUPONT DE NEMOURS; Cr/MgF₂/Al/MgF₂/Cr, pigments having this structure being marketed under the trade name CHROMAFLAIR by FLEX; MoS₂/SiO₂/Al/SiO₂/MOS₂; Fe₂O₃/SiO₂/Al/SiO₂/Fe₂O₃, and Fe₂O₃/SiO₂/Fe₂O₃/SiO₂/Fe₂O₃, pigments having these structures being marketed under the trade name SICOPEARL by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ et TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being marketed under the trade name XIRONA by MERCK (Darmstadt). By way of example, these pigments may be the pigments with a silica/titanium oxide/tin oxide structure marketed under the name XIRONA MAGIC by MERCK, pigments with a silica/brown iron oxide structure marketed under the name XIRONA INDIAN SUMMER by MERCK and pigments with a silica/titanium oxide/mica/tin oxide structure marketed under the name XIRONA CARRIBEAN BLUE by MERCK. Mention may also be made of the INFINITE COLORS pigments from SHISEIDO. Depending on the thickness and the nature of the various layers, various effects are obtained. Thus, with the structure Fe₂O₃/SiO₂/Al/SiO₂/Fe₂O₃, the color changes from green-golden to red-gray for SiO₂ layers from 320 to 350 nm; from red to golden for SiO₂ layers from 380 to 400 nm; from violet to green for SiO₂ layers from 410 to 420 nm; from copper to red for SiO₂ layers from 430 to 440 nm.

By way of example of pigments with a polymeric multilayer structure, mention may be made of those marketed by 3M under the trade name COLOR GLITTER.

Examples of liquid-crystal goniochromatic particles that may be used include those sold by CHENIX, and also that marketed under the trade name HELICONE® HC by WACKER.

Additional Fillers

A composition according to the invention may also comprise at least one additional filler, different to the above-mentioned filter absorbing and/or adsorbing sebum, of organic or inorganic nature, which makes it possible in particular to confer thereon additional properties of mattness, covering power and/or improved stability or staying power.

The filler content may range from 2% to 20% by weight, and in particular from 4% to 12% by weight, with respect to the total weight of said composition.

The term “filler” should be understood to mean colorless or white solid particles of any shape, which are in a form that is insoluble or dispersed in the medium of the composition. Mineral or organic in nature, they make it possible to confer body or rigidity on the composition, and/or softness, and uniformity on the makeup.

The fillers used in the compositions according to the present invention may be of lamellar, globular or spherical form, or in the form of fibers or in any other intermediate form between these defined forms.

The fillers may or may not be coated superficially, and in particular they may be surface-treated with silicones, amino acids, fluorinated derivatives or any other substance that promotes the dispersion and compatibility of the filler in the composition.

As examples of mineral fillers, mention may be made of talc, mica, silica, hollow silica microspheres, kaolin, calcium carbonate, magnesium carbonate, hydroxyapatite, boron nitride, glass or ceramic microcapsules, composites of silica and of titanium dioxide, such as the TSG series marketed by Nippon Sheet Glass.

As examples of additional organic fillers, mention may be made of polyethylene powders, polytetrafluoroethylene (Teflon) powders, lauroyl lysine, hollow polymeric microspheres such as those of polyvinylidene/acrylonitrile chloride, for instance Expancel (Nobel Industries), hexamethylene diisocyanate/Trimethylol hexyllactone copolymer powder (Plastic Powder from Toshiki), silicone resin microbeads (Tospearl from Toshiba, for example), synthetic or natural micronized waxes, metal soaps derived from organic carboxylic acids having 8 to 22 carbon atoms, and preferably from 12 to 18 carbon atoms, for example, zinc stearate, magnesium stearate or lithium stearate, zinc laurate, magnesium myristate, cross-linked elastomeric organopolysiloxane powders coated with silicone resin, in particular silsesquioxane resin, as described for example in the patent U.S. Pat. No. 5,538,793, polyurethane powders, in particular, cross-linked polyurethane powders including a copolymer, wherein said copolymer comprises trimethylol hexyllactone. In particular, it may be a polymer of hexamethylene diisocyanate/trimethylol hexyllactone. Such particles are in particular commercially available, for example under the trade name PLASTIC POWDER D-400® or PLASTIC POWDER D-800® from TOSHIKI, and mixtures thereof.

According to one particular embodiment of the invention, the composition comprises at least one cross-linked elastomeric organopolysiloxane powder coated with silicone resin. The presence of this filler also enables the composition according to the invention to be thickened and/or gelled.

The cross-linked elastomeric organopolysiloxane powder(s) coated with silicone resin may be present in a content ranging from 2% to 12% by weight, advantageously from 4% to 10% by weight and preferably from 7% to 9% by weight with respect to the total weight of said composition.

In particular, mention may be made of cross-linked elastomeric organopolysiloxane powders coated with silicone resin, in particular silsesquioxane resin, as described for example in the patent U.S. Pat. No. 5,538,793. Such elastomer powders are sold under the trade names KSP-100®, KSP-101®, KSP-102®, KSP-103®, KSP-104® and KSP-105® by SHIN ETSU; mention may also be made of cross-linked elastomeric organopolysiloxane powders coated with silicone resin such as hybrid silicone powders functionalized by fluoroalkyl groups, in particular sold under the trade name “KSP-200” by Shin Etsu; or hybrid silicone powders functionalized by phenyl groups, in particular sold under the trade name “KSP-300” by Shin Etsu.

Additives

A cosmetic composition according to the invention may also further comprise any additive normally used in the field in question, for example selected from gums, anionic, cationic, amphoteric or nonionic surfactants, silicone surfactants, gums, resins, dispersants, semicrystalline polymers, antioxidants, essential oils, preservatives, perfumes, neutralizing agents, antiseptics, anti-UV protective agents, cosmetic agents, such as vitamins, hydrating agents, emollients or collagen-protecting agents, and mixtures thereof.

A person skilled in the art can adjust the type and quantity of additives present in the compositions according to the invention by means of routine operations, so that the cosmetic properties and the stability properties sought for these compositions are not affected by the additives.

A cosmetic composition of the invention may in particular be in the form of an anhydrous liquid product, an anhydrous gel, in the form of a stick or in the form of a soft paste. In particular, a cosmetic composition of the invention may be in the form of a liquid or fluid foundation, a hot-pour foundation, a body makeup product, a concealer or an eye shadow, or a makeup base.

A care composition according to the invention may, in particular, be a sun composition, a skincare cream, a serum or a deodorant.

Preferably, the composition according to the invention is in the form of a fluid foundation.

The present invention also relates to a cosmetic treatment method including the application on the face of a composition as defined above.

The present invention also relates to a non-therapeutic makeup and/or skincare method including a step for applying, on the skin, at least one layer of a composition as defined above.

The present invention also relates to a skin makeup method wherein a composition as defined above is applied.

Device

The invention also relates to a packaging and application device of a cosmetic composition as defined above, comprising:
a container containing said cosmetic composition; and
means for dispensing said composition drop by drop.

According to one embodiment, the present invention relates to a packaging and application device of a cosmetic composition as defined above, comprising:
a container containing said cosmetic composition, the container being a flexible-walled tube, and
a dispensing head comprising a dispensing orifice, the device being characterized in that the area having the smallest cross-section in respect of the flow of the composition between the container and the dispensing orifice is between 0.2 mm² and 3 mm², preferably 0.5 mm² and 2 mm², more preferably from 0.8 mm² to 1.7 mm² and more preferentially from 1 mm² to 1.2 mm².

According to one particular embodiment, the device comprises at least one restriction of the flow of the composition between the container and the dispensing orifice, said restriction having a cross-section wherein the area is less than the area of the cross-section of the dispensing orifice.

As such, the present invention also relates to a cosmetic makeup method for keratin materials, particularly skin, comprising the following steps:
the flexible-walled tube of the packaging and application device as defined above is pressed for a controlled drop-by-drop discharge of the cosmetic composition according to the invention, and
the cosmetic composition is spread with a finger, particularly on facial skin, and smoothed until homogenization.

According to a further embodiment, the present invention relates to a packaging and application device of a cosmetic composition as defined above, comprising:
a container, particularly made of glass,
a cosmetic composition according to the invention contained in the container,
a closing element,
a pipette, particularly made of glass, attached to the closing element, and
a compressible chamber connected to the pipette.

Further devices suitable for drop-by-drop application are also described in the international applications WO01/47784 and WO2011/154397.

As such, the present invention also relates to a cosmetic makeup method for keratin materials, particularly skin, comprising the following steps:
the device container is shaken and a pressure is applied to the compressible chamber to take up the liquid composition in the pipette,
the pipette is removed from the container and a further pressure is applied to the compressible chamber to apply the composition in a controlled fashion, for example on the palm of the hand, and
the cosmetic composition is spread with a finger, particularly on facial skin, and smoothed until homogenization.

Throughout the application, the term “comprising a” or “including a” means “comprising at least one” or “including at least one”, unless otherwise specified.

Throughout the above description, unless specified otherwise, the term “between x and y” refers to an inclusive range, i.e. the values x and y are included in the range.

The invention will now be illustrated in the following non-limiting examples. Unless specified otherwise, the % are expressed by weight in relation to the total weight of the composition.

The compositions are prepared using routine cosmetic composition formulation methods.

### EXAMPLES

Compositions according to the invention (foundation) are prepared as described hereinafter.

### Example 1

Fluid foundation example 1 illustrates the use of perlite, in conjunction with a high ethanol content.

This composition comprises the following ingredients:

| | | mass % |
|---|---|---|
| A1 | Isododecane | 9.00 |
| | Disteardimonium hectorite/propylene | 10.00 |
| | carbonate/isododecane (Oct. 3, 1987) sold under | |
| | the reference BENTONE GEL ISD V by Elementis | |
| | Phenyltrimethicone sold under the | 4.00 |
| | reference DC 556 by Dow Corning | |
| | Ethyl hexyl methoxycinnamate | 3.00 |
| A2 | Cyclohexasiloxane | 4.00 |
| | Dimethicone copolyol sold under the | 1.50 |
| | reference KF 6017 by Shin Etsu | |
| | Yellow iron oxide coated with aluminum | 2.98 |
| | stearoyl glutamate | |
| | Red iron oxide coated with aluminum | 0.94 |
| | stearoyl glutamate | |
| | Black iron oxide coated with aluminum | 0.32 |
| | stearoyl glutamate | |
| | Titanium dioxide coated with aluminum | 6.76 |
| | stearoyl glutamate | |
| A3 | Dodecamethylpentasiloxane | 20.65 |
| | Cyclohexasiloxane | 16.60 |
| | Vinyl dimethicone/Methicone Silsesquioxane | 8.00 |
| | cross-polymer sold under the reference | |
| | KSP 100 by Shin Etsu | |
| A4 | Perlite sold under the trade name | 0.25 |
| | OPTIMAT 2550 OR by World Minerals | |
| B | Denatured ethanol at 96° | 11.95 |
| C | Perfume | 0.05 |
| | TOTAL | 100% |

Procedure

The constituents of phase A1 are weighed in the main beaker and stirred, at ambient temperature, with a Rayneri mixer (200 to 300 rpm) for 15 minutes until homogenization.

Phase A2 is prepared separately by grinding three times, in a three-roll mill, the mixture of pigments, dimethicone copolyol and cyclohexasiloxane.

This phase A2 is then added, while stirring, to the Rayneri mixer (400 to 500 rpm) for 10 minutes at ambient temperature.

Phase A3 is prepared separately by dispersing KSP 100 in the mixture of the two oils, while stirring at ambient temperature, with a Rayneri mixer (500 rpm) for 10 minutes until homogenization.

This phase A3 is then added slowly, while stirring at ambient temperature, to the Rayneri mixer (500 rpm) for 10 minutes.

Phase A4 is then added, while stirring, to the Rayneri mixer (400 to 500 rpm) for 10 minutes at ambient temperature.

Rayneri stirring is then reduced to 200-300 rpm and phases B and C are added. After incorporation, the mixture is left under stirring for 5 more minutes at ambient temperature and then packaged without delay.

### Example 2

Fluid foundation example 2 illustrates the use of silica silylate, in conjunction with a high ethanol content.

This composition comprises the following ingredients:

| | | mass % |
|---|---|---|
| A1 | Isododecane | 9.00 |
| | Disteardimonium hectorite/propylene | 10.00 |
| | carbonate/isododecane (Oct. 3, 1987) | |
| | sold under the reference BENTONE GEL | |
| | ISD V by Elementis | |
| | Dimethicone copolyol sold under the | 1.50 |
| | reference KF 6017 by Shin Etsu | |
| | Phenyltrimethicone sold under the | 4.00 |
| | reference DC 556 by Dow Corning | |
| | Ethyl hexyl methoxycinnamate | 3.00 |
| A2 | Dodecamethylpentasiloxane | 20.65 |
| | Cyclohexasiloxane | 12.10 |
| | Vinyl dimethicone/Methicone Silsesquioxane | 8.00 |
| | cross-polymer sold under the reference | |
| | KSP 100 by Shin Etsu | |
| A3 | Cyclohexasiloxane | 4.00 |
| | Yellow iron oxide coated with aluminum | 2.98 |
| | stearoyl glutamate | |
| | Red iron oxide coated with aluminum | 0.94 |
| | stearoyl glutamate | |
| | Black iron oxide coated with aluminum | 0.32 |
| | stearoyl glutamate | |
| | Titanium dioxide coated with aluminum | 6.76 |
| | stearoyl glutamate | |
| A4 | Cyclohexasiloxane | 4.50 |
| | Silica silylate sold under the trade | 0.25 |
| | name Dow Corning VM-2270 Aerogel | |
| | Fine Particles by Dow Corning | |
| B | Denatured ethanol at 96° | 11.95 |
| C | Perfume | 0.05 |
| | TOTAL | 100% |

Procedure

The constituents of phases A1 and A2 are weighed into the main beaker and stirred, at ambient temperature, with a Moritz mixer (3200 rpm) for 20 minutes until homogenization.

Phase A3 is prepared separately by grinding three times, in a three-roll mill, the mixture of pigments and cyclohexasiloxane.

Phase A4 is also prepared separately, in a capsule, mixing cyclohexasiloxane and silica silylate with a spatula, and then added to phase A3 and homogenized by hand.

The mixture of phases A3 and A4 is reground once with the three-roll mill. The ground product (A3+A4) is then introduced into the main beaker containing phases A1+A2 while stirring with a Moritz mixer (3000 rpm, small turbine) for 15 minutes, until homogenization.

Phases B and C are mixed, and the whole is introduced into the main beaker while stirring with a Moritz mixer (2800 rpm, small turbine) for 5 minutes, until homogenization.

The foundation is then packaged without delay.

1. Cosmetic composition, comprising a cosmetically acceptable medium, containing a fatty phase and an aqueous phase,
said aqueous phase comprising between 8% and 40% by weight of one or a plurality of mono-alcohol(s) comprising 2 to 8 carbon atoms, with respect to the total weight of said composition, said composition comprising at least one filler absorbing and/or adsorbing sebum wherein the oil absorption capacity is greater than or equal to 1.5 ml/g, and
said composition comprising water in a quantity less than or equal to 5% by weight with respect to the total weight of said composition.

2. Cosmetic composition according to claim 1, wherein the fatty phase represents 25% to 85% by weight with respect to the total weight of said composition.

3. Cosmetic composition according to claim 1, comprising less than 5% by weight of water with respect to the total weight of said composition.

4. Cosmetic composition according to claim 1, wherein the filler absorbing and/or adsorbing sebum is in the fatty phase.

5. Cosmetic composition according to claim 1, wherein the filler absorbing and/or adsorbing sebum has a BET specific surface area greater than or equal to 300 m2/g.

6. Cosmetic composition according to claim 1, wherein the filler absorbing and/or adsorbing sebum is chosen from the group consisting of silicas, silica silylates, polyamide powders, acrylic polymer powders, perlites, magnesium carbonate and mixtures thereof.

7. Cosmetic composition according to claim 6, wherein the filler absorbing and/or adsorbing sebum is chosen from the group consisting of silicas, acrylic polymer powders, perlites, magnesium carbonate and mixtures thereof.

8. Cosmetic composition according to claim 1, wherein the filler absorbing and/or adsorbing sebum is a perlite powder or a silica powder.

9. Cosmetic composition according to claim 1, wherein the filler absorbing and/or adsorbing sebum is present in a quantity ranging from 0.05% to 20% by weight with respect to the total weight of the composition.

10. Composition according to claim 1, wherein the fatty phase comprises at least one volatile oil and/or at least one non-volatile oil.

11. Cosmetic composition according to claim 1, wherein the fatty phase comprises 40% to 100% by weight of volatile oil(s) with respect to the total weight of the fatty phase, and less than 60% by weight of non-volatile oil(s) with respect to the total weight of the fatty phase.

12. Cosmetic composition according to claim 1, wherein the mono-alcohol is ethanol.

13. Cosmetic composition according to claim 1, further comprising a powder substance, different than the filler absorbing and/or adsorbing serum.

14. Cosmetic composition according to claim 1, comprising a thickener or gelling agent and/or a lipophilic structuring agent.

15. Cosmetic composition according to claim 1, in the form of a fluid foundation.

16. Non-therapeutic makeup and/or skincare method comprising a step for applying at least one layer of a cosmetic composition according to claim 1 onto the skin.

17. Packaging and application device of a cosmetic composition according to claim 1, comprising a container containing said cosmetic composition, and means for dispensing said composition drop by drop.

18. Cosmetic composition according to claim 2, comprising less than 5% by weight of water with respect to the total weight of said composition.

19. Cosmetic composition according to claim 2, wherein the filler absorbing and/or adsorbing sebum is in the fatty phase.

20. Cosmetic composition according to claim 3, wherein the filler absorbing and/or adsorbing sebum is in the fatty phase.
